(19) United States
(12) Patent Application Publication
(10) Pub. No.: US 2022/0322955 A1
(43) Pub. Date: Oct. 13, 2022

(54) APPARATUS TO DIAGNOSE AND TREAT INTRACRANIAL CIRCULATION

(71) Applicants: Mindaugas PRANEVICIUS, Forest Hills, NY (US); Henrikas PRANEVICIUS, Kaunas (LT); Osvaldas PRANEVICIUS, New York, NY (US)

(72) Inventors: Mindaugas PRANEVICIUS, Forest Hills, NY (US); Henrikas PRANEVICIUS, Kaunas (LT); Osvaldas PRANEVICIUS, New York, NY (US)

(21) Appl. No.: 17/715,626

(22) Filed: Apr. 7, 2022

(30) Foreign Application Priority Data
Apr. 7, 2021 (LT) LT2021 515

(51) Int. Cl. A61B 5/03

(52) U.S. Cl. CPC A61B 5/031; A61B 2562/0247

(57) ABSTRACT

Apparatus for the diagnostics and treatment of conditions presenting as intracranial circulation maladies in reliance upon segmental intracranial compartment pressure, which is established from the interdynamics between intra-cranial and extra-cranial circulation, and which relies upon compression of the extra-cranial vascular network in order to: measure cranial inflow and outflow pressure in the intra-extra cranial collateral (e.g., in the network supplied by the supraorbital artery), to estimate intracranial compartment segmental perfusion pressure; temporarily augment intracranial inflow pressure during a period of the compromise (e.g., common carotid cross-clamp during carotid endarterectomy or extracranial stenosis with low-flow state) and redirect extracranial blood-flow intracranially to augment cerebral circulation and/or introduce therapeutic agents or cold blood to the intracranial compartment.

### CROSS-REFERENCE TO RELATED APPLICATIONS

The invention described and claimed herein below also is described in Lithuanian Patent Application No. LT2021 515, filed on Apr. 7, 2021 (“the Lithuanian Patent Application”). The Lithuanian Patent Application, the subject matter and contents of which being incorporated herein by reference, provides the basis for a claim of priority of invention under 35 USC § 119(a)-(d).

### BACKGROUND OF THE INVENTION

This invention is directed broadly to medical systems, devices, and methods for diagnosing and treating medical conditions affecting blood supply to the brain.

The invention is more specifically directed to treatment systems, apparatus, and methods for monitoring of segmental intracranial perfusion pressure, assessing cerebral autoregulation and intra-extracranial blood flow distribution, and diverting extracranial blood flow intracranially for therapeutic purposes based on the monitoring of intra-extracranial blood flow distribution, and assessment of intracranial segmental perfusion pressure.

Cerebral perfusion is determined by the inflow (arterial) and outflow (venous) pressures. Outflow pressure corresponds to the intracranial pressure (ICP), which is intracranial pressure, perfusion pressure is arterial pressure Pa minus ICP; segmental perfusion pressure is intracranial inflow pressure Pd minus ICP. ICP can be measured directly with an intraventricular catheter or with an intracranial pressure sensor. ICP also can be assessed noninvasively from known methods, such as by that disclosed in U.S. Pat. No. 8,998,818, entitled: “Noninvasive Method To Measure Intracranial And Effective Cerebral Outflow Pressure,” Pranevicius, et al. (“the '818 patent”).

The system and method of the '818 patent detect and measure intracranial and regional outflow pressure in reliance upon a device to control occlusion of the jugular venous outflow (“occlusion device”), a device to measure hemodynamic parameters during the controlled occlusion (“measurement device”), a processor in communication with the occlusion and measurement devices that estimates intracranial pressure based on the functional dependence of the intracranial venous outflow on the intracranial pressure and jugular occlusion. The system and method operate to establish a dynamic equilibrium between jugular occlusion and intracranial pressure and displays this pressure estimate on the monitor and/or patient monitoring network.

Alternatively, extra-intracranial blood outflow distribution may be measured using NIRS (near-infrared spectroscopy) and balanced by the external compression cuff. In that case, the external cuff pressure at the equilibrium is displayed as the intracranial pressure, as disclosed in U.S. Pat. No. 8,109,880, entitled: “Noninvasive Method To Measure Intracranial And Effective Cerebral Outflow Pressure,”, Pranevicius, et al. (“the 880 patent”).

As known in the related art, the craniospinal venous system has multiple anastomoses between the jugular veins and vertebral venous plexus. Jugular veins collapse with cervical compression or head elevation when the extrinsic pressure exceeds the venous pressure. The vertebral venous plexus is exposed to intracranial pressure (ICP) and collapses when intracranial pressure exceeds venous pressure. Vertebral venous plexus is not compressed with head elevation or cervical compression, because enclosure in the spinal canal protects veins from the direct effects of atmospheric pressure and cervical compression. Using cervical compression and/or head elevation, blood outflow can be redistributed between jugular veins and vertebral venous plexus, while the degree of cervical compression or head elevation indicates effective cerebral outflow pressure or ICP.

U.S. Pat. No. 10,405,763, entitled: “Devices And Methods For Noninvasive Measurement Of Intracranial Pressure,” describes a method for the indirect measurement of ICP using compression of the eye. The average pressure in the circle of Willis is inflow pressure for the intracranial compartment, which can be measured during the catheterization of the intracranial arteries or as a stump pressure during carotid endarterectomy. This average pressure also can be assessed as a pressure in the ophthalmic artery. (Strauss, A. L., Rieger, H., Roth, F. J. & Schoop, W.; “Doppler Ophthalmic Blood Pressure Measurement In The Hemodynamic Evaluation Of Occlusive Carotid Artery Disease;” Stroke 20, 1012-1015 (1989).

Redundant intracranial vascular supply with multiple intra-extracranial collaterals complicates assessment of cerebral hemodynamics in the presence of extracranial stenosis and predisposes to intra-extracranial blood flow diversion (steal). The adequacy of intracranial perfusion is presumed when a person demonstrates normal neurological function. But such assessment is limited when the patient is anesthetized, sedated, intubated, has pre-existing neurological or psychiatric condition, traumatic brain injury, or is intoxicated.

For that matter, during a carotid endarterectomy, when the carotid artery is cross clamped, the adequacy of cerebral perfusion can be assessed by the carotid stump pressure (Pd). Carotid stump pressure represents inflow pressure in the intracranial compartment at the circle of Willis with values above 40 mmHg considered adequate for cerebral perfusion (Moritz, S., Kasprzak, P., Arlt, M., Taeger, K. & Metz, C.; Accuracy Of Cerebral Monitoring In Detecting Cerebral Ischemia During Carotid Endarterectomy: A Comparison Of Transcranial Doppler Sonography, Near-Infrared Spectroscopy, Stump Pressure, and Somatosensory Evoked Potentials; Anesthesiology 107, 563-569 (2007).

The difference between the pressure at the circle of Willis (Pd) and intracranial pressure (ICP) is segmental intracranial compartment perfusion pressure-driving gradient for the cerebral perfusion: SPPic=Pd-ICP. Currently, Pd is not assessed in the clinical setting (apart from carotid endarterectomy cases), and cerebral perfusion is managed using cerebral perfusion pressure (Cerebral Perfusion Pressure: CPP=Pa-ICP) instead, where (Pa) is systemic pressure. Such an approach does not account for the extracranial pressure gradient Pa-Pd, which can be expressed as the fractional flow reserve (FFR=Pd/Pa<1). FFR 0 corresponds to complete inflow occlusion and FFR 1 means zero inflow resistance.

Pd and FFR was recently measured invasively and can be assessed noninvasively using ophthalmodynamometry doppler (measuring ophthalmic artery pressure) or measuring pressure in the intra-extracranial collateral-supraophthalmic artery using Doppler, photoplethysmography, laser Doppler, or oscillometric techniques.

As Intra-extracranial blood flow distribution depends on the resistances and outflow pressures in the corresponding vascular networks, high inflow resistance and high intracranial outflow pressure-common combination in neurotrauma and stroke leads to poorly predictable compromise of intracranial perfusion. Invention describes (1) means to quantify segmental perfusion pressure of the intracranial compartment (SPPic), (2) means to determine whether SPPic is inadequate (below lower limit of the cerebral autoregulation) and needs be augmented, (3) means to quantify contribution of the extracranial stenosis and intra-extracranial outflow pressure gradient (ICP-Pe) to the SPPic reduction, what allows dynamic SPPic estimation from the systemic pressure Pa and intracranial pressure ICP with the goal maintaining SPPic above lower limit of the autoregulation, (4) means to manipulate intra-extracranial outflow gradient for the purpose of intracranial segmental perfusion pressure SPPic estimation, augmentation and reversal of the intra-extracranial blood flow diversion to augment cerebral blood flow, selectively cool the brain and to introduce therapeutic substances (thrombolytics, anesthetics, etc.), without the need of selective intracranial artery catheterization.

### SUMMARY OF THE INVENTION

The invention overcomes the shortcomings of the related art, such as the prior art systems, method and devices referred to above.

The invention provides for active redistribution of the intra-extracranial flow for the therapeutic purposes, e.g., for cerebral blood flow augmentation and intracranial diversion of the therapeutic substances (cooled blood from the extracranial compartment, thrombolytics, anesthetics, etc.) without the need to catheterize intracranial arteries.

In an embodiment, the invention provides apparatus for diagnosing and treating intracranial circulation deficits through measurement and augmentation of segmental intracranial compartment pressure, established from the interdynamics between intra-cranial and extra-cranial circulation. The inventive apparatus, system and method rely upon compression of the extra-cranial vascular network: 1) to measure cranial inflow and outflow pressure in the intra-extra cranial collateral vascular network (e.g., in the network supplied by the supraorbital artery), and estimate intracranial compartment segmental perfusion pressure; 2) temporarily augment intracranial inflow pressure during a period of the compromise, when reduction of the inflow pressure below lower limit of cerebral autoregulation leads to reduction of the cerebral blood flow and, possible cerebral ischemia. Examples of such conditions include common carotid cross-clamp during carotid endarterectomy or extracranial stenosis with low-flow state during trauma, anesthesia in the sitting position, endovascular stroke treatment) and 3) to redirect extracranial blood-flow intracranially to augment cerebral circulation and/or introduce therapeutic agents or cold blood to the intracranial compartment.

The human skull divides the head into cranial and facial compartments. Blood from the aorta with the mean pressure Pa is supplied to the intracranial compartment via two internal carotid and two vertebral arteries. This aortic blood is distributed to the brain via the circle of Willis with a mean pressure Pd. Intracranial outflow pressure is determined by the intracranial pressure ICP. Segmental perfusion pressure for the intracranial compartment (SPPic=Pd-ICP) determines cerebral perfusion. Segmental perfusion pressure for the extracranial compartment SPPec is determined by the pressure in the external carotid artery Pd and extracranial compartment outflow pressure Pe, which is usually atmospheric pressure: SPPec=Pd-Pe.

The Inventors herein have experimentally tested the relationship between Pa, Pd, ICP, and Pe and intra-extracranial blood flow redistribution: Pranevicius, M., Pranevicius, H. & Pranevicius, O.; Cerebral Venous Steal Equation For Intracranial Segmental Perfusion Pressure Predicts And Quantifies Reversible Intracranial To extracranial Flow Diversion. Sci Rep 11, 7711 (2021).

Intracranial inflow pressure Pd can be measured in the intra-extracranial collateral (e.g., supraorbital artery). To equilibrate supraorbital artery pressure with Pd at the circle of Willis, extracranial contribution via external carotid branches has to be minimized (using manual occlusion or infraorbital cuff). Likewise, intracranial pressure ICP equilibrates with the extracranial venous outflow pressure when the extracranial venous outflow is occluded by the infraorbital cuff. Extracranial venous outflow pressure in the equilibrium with ICP can be measured directly or noninvasively, while the infraorbital cuff is inflated. Also, a lower limit of cerebral autoregulation can be assessed by measuring the correlation between systemic-intracranial inflow pressure gradient (Pa-Pd) and the systemic arterial pressure Pa. Below the lower limit of the autoregulation, cerebral blood flow (and gradient Pa-Pd over inflow resistance) decreases with lower arterial pressure Pa, while above this limit cerebral blood flow and corresponding Pa-Pd gradient stay the same, as cerebral blood flow is maintained constant, when autoregulation is intact

During the inflow pressure measurement procedure, an infraorbital cuff is applied to control extracranial outflow pressure Pe without compromising upper airway patency or intracranial inflow. The infraorbital cuff is inflated above systemic pressure occludes extracranial network and intracranial inflow pressure is measured. Inflating the infraorbital cuff above venous pressure equilibrates outflow pressure with intracranial outflow pressure, which is measured extracranially.

Decreasing the infraorbital cuff pressure below diastolic pressure increases pressure in the intra-extracranial collateral, redirects blood flow intracranially and augments cerebral blood flow. Increasing Pe with the infraorbital cuff diverts blood flow intracranially and augments cerebral blood flow, raising Pd. Augmentation of cerebral blood flow may be used during critical periods of cerebral perfusion, for example, including, but not limited to carotid cross-clamping during carotid endarterectomy, an acute phase of a stroke, post-cardiopulmonary resuscitation, post-head trauma treatment, thrombolysis treatment, during endovascular stroke treatment, during shock and during anesthesia in the beach-chair position.

Therapeutic agents (cold for selective brain cooling, thrombolytics, chemotherapy, antibiotics, anesthetics, neuroleptics, seizure medications, etc.) can be introduced intracranially using extra-intracranial blood flow redirection preferentially increasing the concentration of the therapeutic agent in the intracranial compartment without the need for selective intracranial artery catheterization.

To measure blood flow distribution between intracranial and extracranial compartments, blood flow must be measured in the internal carotid artery (CBF) and external carotid artery (Q_ec), using MRI or CT angiography with flow quantification or with ultrasound Doppler flowmetry. Correlation of internal carotid artery flow with arterial blood pressure can be used to assess cerebral autoregulation: Chi, N F; Ku, H L; Wang, C Y; Liu, Y; Chan, L; Lin, Y C; Peng, C K; Novak, V; Hu, H H; Hu, C J; Dynamic Cerebral Autoregulation Assessment Using Extracranial Internal Carotid Artery Doppler Ultrasonography; Ultrasound Med Biol. 2017 July; 43(7):1307-1313. Blood flow can be redistributed to the internal carotid artery during induced hypotension, as verified by Ogoh, S; Lericollais, R; Hirasawa, A; Sakai, S; Normand, H; Bailey, D M; Regional Redistribution Of Blood Flow In The External And Internal Carotid Arteries During Acute Hypotension; Am J Physiol Regul Integr Comp Physiol; 2014 May 15; 306(10):R747-51.

The simultaneous registration of blood flow in the internal and external carotid arteries in combination with arterial blood pressure provides for assessing cerebral autoregulation status, as well as assessing ICP noninvasively by estimating intracranial blood outflow model parameters. Kashif, F M; Verghese, G C; Novak, V; Czosnyka, M; Heldt, T; Model-based Noninvasive Estimation Of Intracranial Pressure From Cerebral Blood Flow Velocity And Arterial Pressure; Sci Transl Med., 2012 Apr. 11; 4(129):129ra44. With infraorbital cuff pressure Pe, manipulation accuracy of model parameter estimation can be verified, as demonstrated in: Pranevicius, M., Pranevicius, H. & Pranevicius, O.; Cerebral Venous Steal Equation For Intracranial Segmental Perfusion Pressure Predicts And Quantifies Reversible Intracranial To extracranial Flow Diversion. Sci Rep 11, 7711 (2021).

### BRIEF DESCRIPTION OF THE DRAWING FIGURES

Further features and advantages of the invention will become apparent from the description of embodiments that follows, with reference to the attached figures, wherein:

FIG. 1. depicts a prior art neck cuff used to divert jugular venous outflow into the craniospinal venous plexus with equilibrium pressure equal to the cerebral outflow pressure (ICP);

FIG. 2. Depicts apparatus to measure systemic arterial pressure in the brachial artery (Pa), measure and augment intracranial inflow pressure in the supraorbital artery (Pd), calculate fractional flow reserve (FFR=Pa/Pd) and divert blood flow intracranially by increasing infraorbital cuff pressure (Pe), in accordance with the invention;

FIG. 3. Depicts a method to divert and augment collateral blood flow: selective compression, embolization, infusion of fluids and/or vasopressors, surgical anastomosis.

FIG. 4. Depicts apparatus for the selective cerebral cooling with the enhanced scalp cooling using intermittent negative pressure and infraorbital cuff to divert cooled blood from the scalp intracranially;

FIG. 5. Depicts apparatus to determine intra-extracranial blood flow distribution in the internal and external carotid arteries with variable extracranial occlusion pressure Pe; and

FIG. 6 prior art depicts the common carotid artery and its principal extracranial and intracranial branches.

### DETAILED DESCRIPTION OF THE INVENTION

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

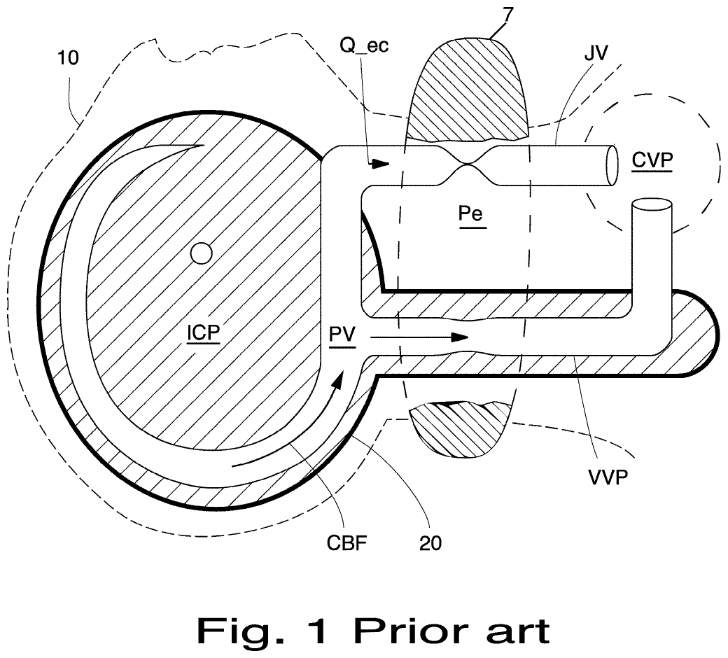

FIG. 1. depicts the use of a prior art neck cuff 7 on a patient to apply pressure to divert jugular venous outflow (JV) into the craniospinal vertebral venous plexus (VVP) with equilibrium pressure equal to the cerebral outflow pressure (ICP), as disclosed in U.S. Pat. No. 8,109,880, to Pranevicius, et al. The reference describes that jugular veins iv can be occluded (22) with an inflatable cervical cuff 7 and equilibrium pressure PV is measured in the head or cervical vein. This occlusion pressure PV represents effective outflow pressure (ICP if ICP is higher than CVP).

To measure PV with iv occlusion, pressure in the head or cervical vein is measured and cervical cuff 7 is gradually inflated. Vein pressure PV plateau when further cuff inflation does not increase venous pressure is displayed as PV_OCCLUSION. Cuff inflation is limited to a maximum safe cuff pressure P_CUFF_MAX, which is selected below diastolic arterial pressure and inspiratory airway occlusion pressure. P_CUFF_MAX may be selected as 20 mmHg (ICP treatment threshold) or higher. If initial PV is high and does not increase with extrinsic compression, effective cerebral outflow pressure is said to be determined by CVP, not the ICP. If PV increases with P_CUFF inflation but does not reach the plateau at P_CUFF_MAX, the effective outflow pressure or ICP is displayed as higher than P_CUFF_MAX.

Apparatus comprises means to compress neck veins 7 (like inflatable or liquid filled cervical tourniquet with pressure P_CUFF) and means to register blood flow/pressure and/or volume in the jugular veins and extrajugular vertebral venous plexus (like Doppler ultrasound or B-mode with color Doppler, or pletysmogram or, manometry—not shown). Q_ec and P_e are extracranial blood flow and extracranial outflow pressure, respectively. The unlabeled circle (just above ICP in FIG. 1 is a hydrostatic reference point at external acoustic meatus.

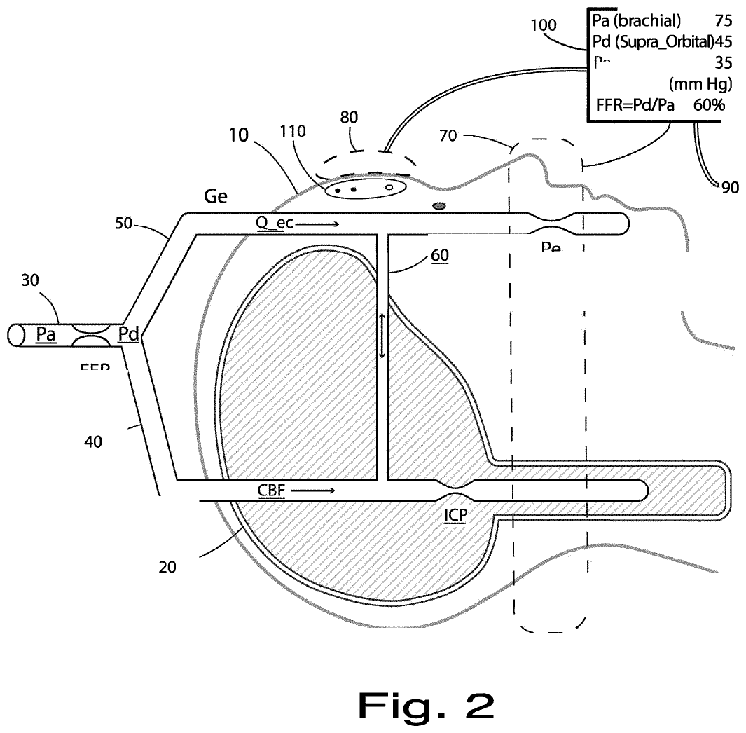

FIG. 2 depicts controller (or microcontroller) 100, which may be a microprocessor with a memory for storing software for implementing the inventive methods) and a display device configured to measure systemic arterial pressure in the brachial artery (Pa, displayed 75 mmHg), to measure and augment intracranial inflow pressure in the supraorbital artery (Pd, displayed 45 mmHg), calculate fractional flow reserve (FFR=Pa/Pd, displayed 45/75=60%) and divert blood flow intracranially by increasing infraorbital cuff pressure (Pe, displayed 35 mmHg).

The apparatus/microprocessor 100 measures the systemic arterial pressure Pa in cooperation with brachial cuff 90 (not shown in FIG. 2), calculates supraorbital arterial (intracranial inflow) pressure Pd using supraorbital cuff 80 and sensor 110 to (as cuff pressure Pd, when maximal oscillometric oscillation in the cuff 80, peak photoplethysmographic oscillation, initial ultrasound or laser Doppler flow signal is obtained), calculates the supraorbital venous (intracranial outflow) pressure ICP in cooperation with the supraorbital cuff 80, infraorbital cuff 70 and sensor 110, as lowest equilibrating infraorbital cuff pressure Pe, which causes facial venous diversion intracranially detected by the supraorbital cuff 80 and/or sensor 110, calculates fractional flow reserve (FFR=Pa/Pd), and controls extracranial outflow pressure Pe to divert extracranial (10) blood flow Q_ec intracranially (20) in cooperation with a infraorbital cuff (70). That is, apparatus/microprocessor 100 facilitates measurement of and/or controls the external pressures in the supraorbital (80), brachial (90) and infraorbital (70) cuffs, estimates and displays said pressures, dynamically estimates segmental perfusion pressure for the intracranial compartment (SPPic=Pd-ICP), assesses status of cerebral autoregulation by correlating Pa-Pd (inflow gradient, proportional to the cerebral blood flow) to systemic arterial pressure Pa.

When the infraorbital cuff 70, which is applied to the facial head compartment 10 below the orbit, is inflated with the pressure Pe to exceed systemic arterial pressure Pa, the extracranial vasculature is occluded, extracranial blood flow Q_ec stops and the Pd measured at the supraophthalmic artery represents the circle of Willis pressure.

When the extracranial outflow pressure Pe decreases below the systemic (mean) arterial pressure in the brachial artery Pa, extracranial inflow resumes, while outflow is occluded. In this case, the extracranial outflow pressure Pd and the segmental perfusion pressure for intracranial compartment SPPic is augmented according to the formula derived from the circuit analysis:

SPPic=Pd-ICP=CPP*FFR-Ge*(1-FFR)*(ICP-Pe)

where Pa is the systemic arterial pressure (measured in the brachial artery), Pd is the intracranial compartment inflow pressure measured in the supraorbital artery, ICP is the intracranial (outflow) pressure, FFR is the fractional flow reserve (FFR=Pd/Pa), Ge is the relative extracranial conductance, and Pe is the extracranial outflow pressure. When extracranial outflow pressure Pe is elevated to occlude extracranial venous outflow and then gradually decreased, once Pe decreases below ICP, venous outflow resumes via the extracranial pathway and extracranial tissue congestion is relieved as extracranial venous pressure falls below ICP, which is registered using plethysmography. Level of Pe when this occurs corresponds to ICP.

ICP can also be estimated measuring venous pressure in the extracranial venous collaterals canulating superficial scalp veins (e.g., supraorbital, superficial temporal) or by retrograde canulation of the external jugular vein, when extracranial outflow is partially obstructed and redirected intracranially (Pe>ICP).

After a series of systemic (mean) arterial pressure Pa measurements, intracranial inflow pressure Pd measurements and cerebral outflow pressure ICP measurements are obtained, fractional flow reserve (FFR) and relative extracranial conductance Ge is estimated from the segmental perfusion pressure for intracranial compartment SPPic equation using least squares method. With estimated FFR, Ge and ICP, SPPic_estimated is predicted from the Pa and Pe. These parameters then are used to monitor and augment SPPic dynamically from the Pa and ICP using formula

SPPic=Pd-ICP=CPP*FFR-Ge*(1-FFR)*(ICP-Pe).

Alternatively, the relative extracranial conductance Ge is assessed from intracranial-extracranial blood flow distribution using MRI, CT or Doppler ultrasound mapping of internal (40) and external (50) carotid arteries. Using fluid dynamic model applied to the morphological 3D model, parameters FFR, ICP, Pd can be estimated using a Kalman filter algorithm from the arterial pressure Pa, extracranial outflow pressure (measured at the infraorbital cuff 70) Pe and relationship of CBF and Q_ec with arterial pressure Pa.

FIG. 2 presents an abstracted version of flow redistribution in the collateral network with common inflow Thevenin equivalent. As shown, element 30 represents common inflow-aorta and all the collaterals. Element 60 represents supraorbital artery-distal extra-intracranial collateral via the ophthalmic artery

The lower limit of cerebral autoregulation can be assessed by measuring the correlation between systemic-intracranial inflow pressure gradient (Pa-Pd) and systemic arterial pressure Pa. Below the lower limit of the autoregulation, cerebral blood flow (and gradient Pa-Pd over inflow resistance) decreases, while above this limit. cerebral blood flow and corresponding Pa-Pd gradient stay the same (statistical hypothesis that correlation coefficient ρ (Pa, Pa-Pd)>0 is rejected). Pa is independent and Pa-Pd (gradient) is measured. ρ denotes correlation function. Alternatively, the lower limit of the cerebral autoregulation can be established from the CBF/Q_ec distribution. Below the lower limit of autoregulation, decreasing blood pressure Pa decreases both CBF in the internal carotid artery (40) and Q_ec via external carotid artery (50). Above lower limit of autoregulation, Q_ec decreases more than CBF when arterial pressure Pa decreases.

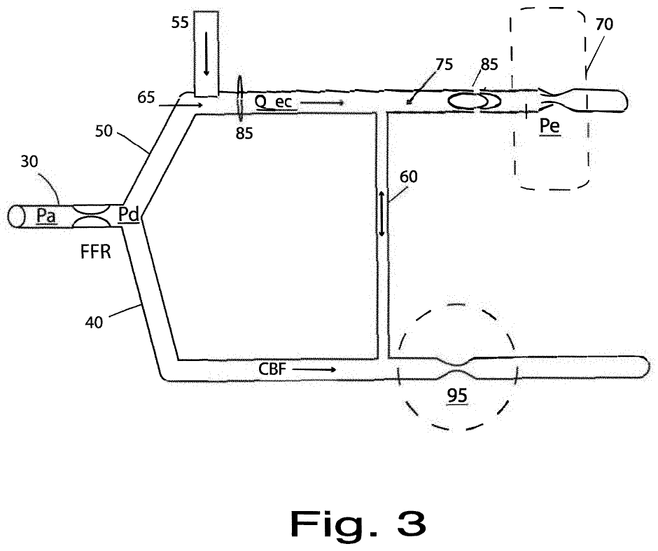

FIG. 3 depicts a method of ischemia treatment according to the invention. The FIG. 3 arrangement provides for inducing reverse steal comprising the steps of: 1) Investigating vascular supply of the ischemic area 40 and identifying blood vessels which also supply blood flow to the adjacent areas (parallel channels 40 and 50), which correspond to the intracranial and extracranial circulation in FIG. 2, identify downstream segments of the parallel channel or channels 50 distal to the take-off of anastomotic connections feeding the ischemic area 40; and 3) selectively increasing pressure at the “Y” take-off of anastomotic connection 50. The takeoff is the common carotid artery. Since there is a left and right carotid, the “Y: takeoff represents the Thevenin inflow equivalent.

Various acts, methods (including reliance up various means) to selectively increase pressure at the anastomotic take-off include but are not limited to: 1) implementing a surgical anastomosis (indicated by element 55 in FIG. 3) to increase inflow (e.g., extra-intracranial arterial bypass; 2) and infusing of fluid or perfusion with blood via antegrade (indicated by arrow 65) or retrograde (indicated by arrow 75) catheter; 3) infusing of vasopressor into downstream segment (at 65): 4) ligating of downstream segment (at 65); 5) using a balloon to occlude or partially occlude a downstream segment; 6) causing an embolization of the downstream segment (indicated at 85); 7) coiling the downstream segment (at 85); and 8) external compressing the downstream segment including by the physical means to induce focal tissue edema (using a cuff at location 95 in FIG. 3).

Acts or methods 1 and 2 (identified above) can be applied anywhere in the secondary channel 50 which is collateral to the network supplying area of interest. Acts or methods 3-8 can be applied distal to the anastomotic take-off in arterial, microcirculatory, or venous segments of the secondary channel.

While as stated herein that the invention includes cerebral blood flow augmentation, the person of ordinary skill in the art should recognize that application of the inventive principles is not limited to the cerebral circulation. The above methods 1-8 may be used for other treatments that might benefit from the alternative blood supply, enabled by the inventive acts disclosed herein. For example, the inventive principles exemplified above could be used for the treatment of ischemia in the myocardium or any other vascular bed where reverse steal (blood flow from the collateral vascular network) can be augmented by selectively increasing pressure at the anastomotic take-of.

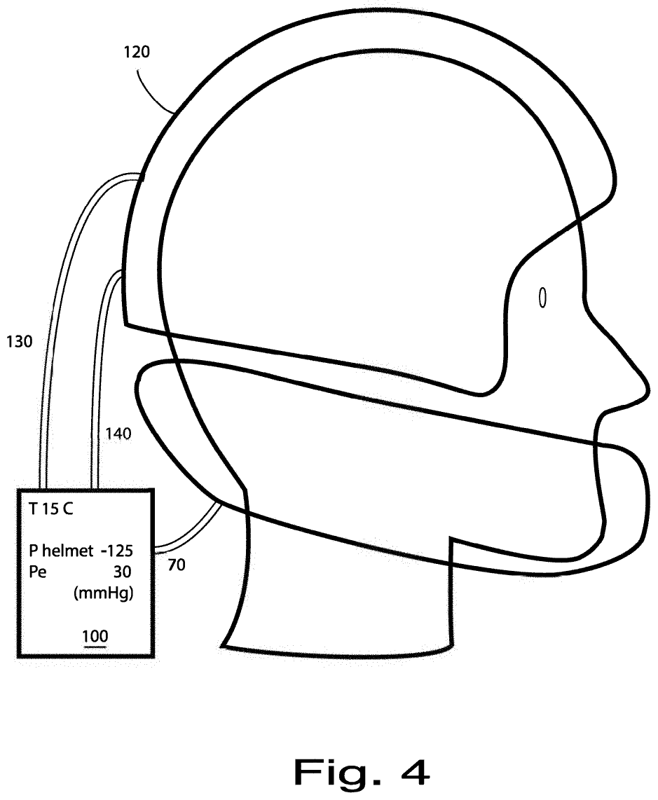

FIG. 4, for example, depicts apparatus for selective cerebral cooling. To implement the act or method of cerebral cooling, a helmet 120 is applied to the head which has hoses for the cooling (130) and helmet pressure control (140). Scalp, and blood in the scalp is cooled by the helmet 120, with helmet temperature and pressure controlled by the microprocessor/controller 100. Intermittent negative pressure enhances scalp blood flow and volume, which is diverted intracranially by the infraorbital cuff 70 controlled by the controller 100.

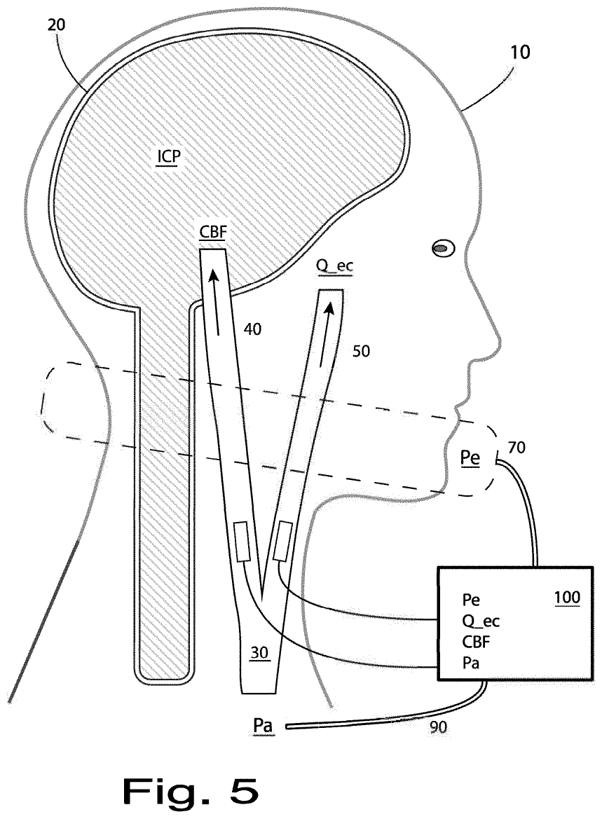

FIG. 5 depicts reconstitution of the cerebral hemodynamic parameters (ICP, Ge, FFR, Pd, and status of the cerebral autoregulation) using arterial pressure Pa, infraorbital pressure Pe and distribution of blood flow via internal (40) and external (50) carotid arteries as an input into model-based parameter assessment using Kalman filter.

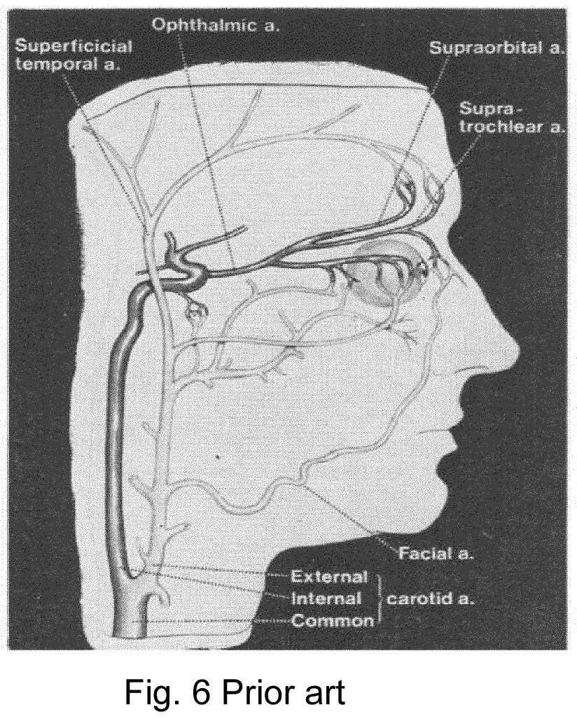

FIG. 6 is a prior art diagram depicts vascular blood supply to the head, intracranially and extracranially.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

### LIST OF NUMERICAL IDENTIFIERS AND SYMBOLS

10 facial (extracranial) head compartment
20 intracranial head compartment
30 common (extracranial) arterial craniofacial inflow
40 intracranial blood supply (internal carotid artery)
50 extracranial blood supply (external carotid artery)
55 surgical anastomosis to augment collateral inflow
60 distal intercompartmental anastomosis (supraophthalmic artery)
65 antegrade catheter
7 neck cuff (PRIOR ART FIG. 1)
70 means for the extracranial outflow pressure control-infraorbital cuff (FIG. 2)
75 retrograde catheter in the collateral pathway
80 supraorbital cuff to measure supraorbital pressure
85 balloon for occlusion of the collateral outflow
90 brachial cuff
95 external pressure-focal tissue edema (equivalent to ICP in the intracranial compartment)
100 controller/microprocessor, with memory and display device or monitor
110 sensor to measure parameter related to the blood volume and/or flow
120 helmet for use in implementing cerebral cooling.
130 hoses for scalp cooling
140 hose to control pressure in the helmet for intermittent scalp blood pooling
p correlation coefficient
CBF cerebral blood flow
CBF/Q_ec ratio of intracranial (CBF) to extracranial (Q_ec) blood flow
CVP central venous pressure
FFR fractional flow reserve FFR=Pd/Pa
Ge relative extracranial conductance
ICP intracranial pressure (effective intracranial outflow pressure)
JV jugular vein
Pa systemic (mean) arterial pressure measured in the brachial artery
Pd intracranial inflow pressure measured in the supraorbital artery
Pe extracranial outflow pressure
PV venous blood pressure
Q_ec extracranial blood flow
SPPic segmental perfusion pressure for intracranial compartment
SPPec segmental perfusion pressure for extracranial compartment
VVP craniospinal Vertebral Venous Plexus

What is claimed is:

1. A system for measuring and augmenting segmental perfusion pressure in the intracranial compartment based on the interdynamics between the intracranial and extracranial circulations, the system comprising:
a processor with a display device and a memory for storing computer-readable instructions;
a brachial cuff or other means to measure systemic arterial pressure (Pa);
a supraorbital cuff or other means to measure supraorbital arterial pressure (Pd);
an infraorbital cuff or other means to control extracranial outflow pressure (Pe), including selectively occluding extracranial outflow;
wherein the processor calculates segmental perfusion pressure, SPPic=Pd-ICP, where ICP is intracranial outflow pressure and Pd is intracranial inflow pressure.

2. The system of claim 1, further comprising means for detecting intra-extracranial blood flow distribution.

3. The system of claim 1, further comprising the processor controlling to divert extracranial blood flow (Q_ec) intracranially

4. The system of claim 1, further comprising a sensor for sensing the supraorbital arterial pressure.

5. The system of claim 1, wherein the processor measures systemic pressure (Pa), the supraorbital pressure (Pd), the cerebral outflow pressure (ICP), displays said pressures on the display device, and dynamically estimates segmental perfusion pressure for the intracranial compartment (SPPic=Pd-ICP).

6. The system of claim 4, wherein the processor assesses a status of cerebral autoregulation by correlating the difference between the systemic pressure (Pa) and the supraorbital pressure (Pd), gradient proportional to blood flow (Pa-Pd) to systemic pressure Pa.

7. The system of claim 4, processor estimates the distribution and the relative extracranial conductance (Ge) to calculate the segmental intracranial perfusion pressure (SPPic=Pd-ICP).

8. The system of claim 7, wherein the segmental intracranial perfusion pressure (SPPic) is calculated by the formula:
SPPic=Pd-ICP=(Pa-ICP)*FFR-Ge*(1-FFR)*(ICP-Pe),
where Pd is the intracranial perfusion pressure, where ICP is the cerebral outflow pressure, where Pa is the systemic arterial pressure, where FFR is the fractional flow reserve, Ge is relative external conductance and Pe is the extracranial outflow pressure.

9. The system of claim 8, wherein the relative external conductance Ge, the fractional flow reserve FFR are estimated from the intracranial perfusion pressure Pd, whereby the segmental intracranial perfusion pressure is estimated as a result of subtracting the cerebral outflow pressure ICP from the systemic arterial pressure Pa.

10. The system of claim 1, wherein pressure is measured in the supraorbital artery, other branches of the external carotid arteries, an ophthalmic artery, and/or corresponding capillary and venous networks.

11. system of claim 1, wherein pressure in an intra-extracranial collateral is measured using pulse propagation time to the branch of internal and/or external carotid arteries;

12. The system of claim 1, wherein pressure is measured in the intra-extracranial collateral network by applying variable external pressure (positive or negative), with optional superimposed extrinsic oscillation, to facilitate noninvasive estimation of the arterial (systolic, mean, diastolic) and venous pressures.

13. The system of claim 1, wherein pressure is measured in the venous portion of the intra-extracranial collateral network, which corresponds to the intracranial (outflow) pressure ICP when extracranial vascular network is partially compressed.

14. A system for measuring and augmenting segmental perfusion pressure in the intracranial compartment based on the interdynamics between the intracranial and extracranial circulations, the system comprising:
a processor with a display device and a memory for storing computer-readable instructions;
an infraorbital cuff or other means to control extracranial outflow pressure (Pe), including selectively occluding extracranial outflow; and
means for detecting intra-extracranial blood flow distribution;
wherein the processor calculates segmental perfusion pressure, SPPic=Pd-ICP, where ICP is intracranial outflow pressure and Pd is intracranial inflow pressure; and
wherein intra-extracranial blood flow distribution is assessed by the magnetic resonance, computer tomography or ultrasound doppler of vessels supplying cranial and facial compartments (internal and external carotid arteries) with intra extracranial blood flow distribution, arterial pressure, morphological data, and infraorbital cuff pressure data used to estimate intracranial pressure ICP, FFR and status of the cerebral autoregulation

15. A system for redirecting extracranial blood flow intracranially using extra-intracranial blood flow diversion, the system comprising:
means for effecting extra-intracranial blood flow diversion; and
means for introducing an extracranial therapeutic agent intracranially using the extra-intracranial blood flow diversion.

16. The system of claim 15, further comprising:
means for augmenting cerebral blood flow, including providing cerebral protection in reliance upon the augmented cerebral blood flow using the extra-intracranial blood flow diversion.

17. The system of claim 16, further comprising means for selective brain cooling using the extra-intracranial blood flow diversion.

18. The system of claim 15, wherein the means for controlling extra-intracranial blood flow diversion includes an infraorbital cuff or other means to control occlusion pressure without impeding respiration.

19. The system of claim 15, wherein the means for controlling extra-intracranial blood flow diversion includes a brachial cuff or other means to control extracranial occlusion.

20. The system of claim 19, wherein the means for controlling extra-intracranial blood flow diversion relies upon occlusion implemented by one or more of the following: a cuff, a tourniquet, a compression dressing, an elastic garment, a pneumatic suit and a pneumatic compression device for the selective compression of the extracranial arteries and/or veins;

21. The system of claim 15, wherein the therapeutic agent may be any of cold blood, a thrombolytic agent, an anesthetic agent, an antibiotic agent, a chemotherapeutic agent, an antiepileptic agent and a neuroleptic agent.

22. The system of claim 15, wherein the therapeutic agent is diverted intracranially from the right radial artery retrogradely via the brachiocephalic trunk, using a brachial tourniquet to block the antegrade blood flow in the right arm;

23. The system of claim 15, wherein a volume of the blood in the scalp and heat transfer from the head to the cooling device is enhanced by constant or intermittent external positive or negative pressure.

24. The system of claim 15, wherein the extra-intracranial blood flow diversion and an intracranial inflow pressure P9 augmentation are used during cerebral low-flow states or when a patient is at risk for low-flow, including a carotid cross-clamp during carotid endarterectomy, acute stroke, endovascular interventions, shock, head trauma, and/or anesthesia while the patient under treatment is in a sitting position, post resuscitation care.

25. A method for calculating segmental intracranial compartment perfusion pressure (SPPic) of an intracranial compartment of a patient under treatment, to assess cerebral autoregulation, the SPPic measured according to the following formula:
Pd-ICP=(Pa-ICP)*FFR-Ge*(1-FFR)*(ICP-Pe),
where Pa is systemic arterial pressure in the aorta, Pd is intracranial compartment inflow pressure, ICP is intracranial (outflow) pressure FFR is fractional flow reserve, or Pd/Pa, Ge is relative extracranial conductance, and Pe is extracranial outflow pressure, the method including steps of:
measuring the intracranial compartment inflow pressure (Pd);
measuring the intracranial (outflow) pressure (ICP);
calculating the difference between the intracranial compartment inflow pressure (Pd) and the intracranial outflow pressure (ICP).

26. The method of claim 25, wherein the step of calculating includes calculating the fractional flow reserve (FRR), the relative extracranial conductance (Ge), measuring the extracranial outflow pressure (Pe), calculating a difference between the systemic arterial pressure in the aorta and the intracranial outflow pressure, and multiplying that difference times the fractional flow reserve (FFR) and subtracting therefrom a mathematical product of the relative extracranial conductance (Ge) times a difference between 1 and the fractional flow reserve (FFR) times a difference between the intracranial (outflow) pressure (ICP) and the extracranial outflow pressure (Pe).

27. The method of claim 25, further comprising segmentally augmenting perfusion pressure of the intracranial compartment in reliance upon the difference between the intracranial compartment inflow pressure (Pd) and the intracranial outflow pressure (ICP).

28. The method of claim 27, further comprising redirecting the extracranial blood flow intracranially to effect the segmental augmental perfusion.

29. A non-transitory computer readable medium, comprising a set of computer-reading instructions that upon processing by a computer processor with a memory implement the method claim 25.
